(19) United States
(12) Patent Application Publication
(10) Pub. No.: US 2022/0193297 A1
(43) Pub. Date: Jun. 23, 2022

(54) APPARATUS AND METHOD FOR SANITIZING AIR AND SURFACES

(71) Applicant: Pioneer Valley Hotel Group, Inc., Springfield, MA (US)

(72) Inventors: Shardool Parmar, Hadley, MA (US); Richard L. Miller, Needham, MA (US); Brian Hyunsuk Yoo, Arlington, MA (US)

(73) Assignee: Pioneer Valley Hotel Group, Inc., Springfield, MA (US)

(21) Appl. No.: 17/132,280

(22) Filed: Dec. 23, 2020

(51) Int. Cl. A61L 9/20; A61L 2/26; A61L 2/10

(52) U.S. Cl. CPC A61L 9/20; A61L 2/26; A61L 2209/12; A61L 2202/11; A61L 2209/14; A61L 2/10

(57) ABSTRACT

An apparatus and method for treating air and surfaces for sanitizing or other cleaning purposes. Air can be filtered and/or treated with UV illumination, and surfaces can be treated with UV illumination, e.g., to oxidize contaminants. Doors on an apparatus housing can be moved from a closed position, in which UV illumination cannot exit the housing, to an open position in which UV illumination can exit the housing to treat nearby surfaces. The doors can be retracted within the housing, and/or can impede air flow along an air path to filters in the housing. Opening of the doors can expose an air flow path through the door opening and to filters.

### BACKGROUND

### 1. Field of Invention

This invention relates to cleaning air and surfaces, such as by filtering impurities from the air and/or exposing air and surfaces to germicidal ultraviolet light.

### 2. Related Art

Residential, commercial and industrial spaces can have atmospheres that are contaminated with dust, grease, microbes or other materials that cause discomfort or health hazards, for example, to people occupying those spaces. Conventional air cleaning technologies filter the air with materials that trap or otherwise capture materials. Other technologies expose air and surfaces to ultraviolet (UV) light to oxidize or otherwise decompose microbes and other materials.

### SUMMARY OF INVENTION

In one aspect, the subject matter disclosed herein relates to methods and assemblies for treating air to remove impurities. Such impurity removal may involve one or more of a treatment to sanitize, filter, decontaminate, deodorize, purify, condition, heat, humidify, and/or dry an atmosphere, for example. In one aspect, the subject matter herein relates to methods and assemblies for treating surfaces with UV illumination, e.g., to clean the surfaces by oxidizing, decomposing or otherwise neutralizing microbes or other contaminants on the surfaces. Such methods and assemblies may employ a fan or other air mover to draw air through the system, a particulate filter to remove aerosols and particulate matter from the air, a UV illumination source to sanitize air and/or surfaces, and a controller to control components such as the UV illumination source and/or air mover based on the inputs from sensed conditions, user input or other operational parameters. In some embodiments, a sanitizer apparatus or method can be deployed at a ceiling of a room and arranged to draw air in, treat the air such as by removing volatile organic compounds (VOCs) and/or inactivating microbes such as viruses and/or bacteria, and return the treated air to the room, as well as emit UV illumination to expose surfaces in the room to germicidal or otherwise decontaminating UV light. One sanitizing approach involves capturing and/or converting odors, gases and/or other contaminants into harmless compounds that are not noticed by or cause harm to occupants in the room.

In some embodiments, a sanitizer apparatus includes a housing having an air inlet, an air outlet, and an air mover arranged to move air along an air flow path in the housing from the air inlet to the air outlet. A filter can be arranged in the air flow path and configured to remove particulate material from air moving along the air flow path, e.g., by trapping, oxidizing or otherwise removing material from the air. A UV illumination source can be arranged in the housing and configured to emit UV illumination suitable to sanitize air and/or surfaces exposed to the UV illumination. In some cases, the UV illumination can include germicidal wavelengths to neutralize microbes such as viruses and bacteria on surfaces near the apparatus and/or in air. A door can be mounted at a door opening of the housing and configured to move between a closed position in which the door is configured to block UV illumination emitted by the UV illumination source from exiting the housing and an open position in which the UV illumination is permitted to exit the housing through the door opening.

In some embodiments, in the open position the door is positioned within the housing, e.g., the door can move from a closed position in which the door is exposed to an open position in which the door is positioned inside of the housing. Positioning the door within the housing in an open position can provide different features. In some embodiments, in the closed position the door can be positioned away from an air flow path, while in the open position the door can be positioned to impede flow of air along the air flow path. For example, the door in the open position can be positioned within the housing between the air inlet and the filter so as to block or impede flow from the air inlet to the filter. In some embodiments, positioning the door inside of the housing can help prevent the door from blocking or otherwise interfering with UV illumination exiting the door opening. For example, the door can be completely retracted into the housing so that the door does not interfere with UV light exiting the door opening.

In some embodiments, movement of a door from the closed position to the open position can cause flow of air along the air flow path to be impeded. As noted above, this can be done by positioning the door at least partially in an air flow path or otherwise to impede air flow (e.g., by positioning the door outside of the housing and over an air inlet). In some embodiments, an element such as a panel, louver, shutter, etc. can be arranged so that the element impedes air flow along an air flow path based on movement of the door to the open position. As an example, a panel or other element can be coupled to the door so that when the door moves to the open position, the panel moves into the air flow path or otherwise is arranged to impede air flow. As another example, movement of the door to the open position can cause a shutter to close an air inlet, thereby blocking flow in through the air inlet. This arrangement can cause air to be drawn into the housing in other ways, e.g., so at least some air drawn into the housing is exposed to UV illumination while being drawn toward the filter. For example, in the closed position the door can be configured to impede air flow into the door opening, but in the open position the door can be positioned to permit flow of air through the door opening and to a filter. Air entering the door opening and flowing to the filter can be exposed to UV illumination and sanitized by the UV illumination. Impeding flow of air along alternate air flow paths (e.g., from an air inlet to a filter) can help encourage flow through the door opening and into the housing. In some cases, air flowing through the door opening can enter into a gap which can be positioned between an inner side of the door and a reflector for the UV illumination source. Air flow through the door opening and into the gap can be driven by the air mover, and air flow into the gap can pass to the filter, e.g., after exposure to UV illumination.

In some embodiments, the housing includes a plurality of openings that form the air inlet where each of the openings extends from an outer side of the housing to an inner side of the housing. In the open position, the door can be positioned adjacent the inner side of the housing and the plurality of openings, e.g., between the plurality of openings and the filter.

In some embodiments, the door is a first door, and the apparatus further includes a second door mounted at a door opening of the housing and configured to move between a closed position in which the second door is configured to block UV illumination emitted by the UV illumination source from exiting the housing, and an open position in which the UV illumination is permitted to exit the housing through the door opening. Thus, the first and second doors can be arranged to close the door opening when the first and second doors are in the closed position and to open the door opening when the first and second doors are in the open position. For example, in the open position, the first and second doors can be configured to permit flow of air through the door opening and to a filter, and in the closed position, the first and second doors are configured to impede flow of air through the door opening. The first and second doors can move away from each other along a linear path and/or along a rotational or pivotal path when moving from the closed position to the open position. In some embodiments, the filter is a first filter, the air inlet is a first air inlet and the air flow path is a first air flow path, and the apparatus further includes a second filter, a second air inlet and a second air flow path. In the closed position the second door is positioned away from the second air flow path and in the open position the second door is positioned to impede flow of air along the second air flow path from the second air inlet to the second filter. Thus, the first and second doors can be configured to impede air flow along respective first and second air flow paths when in the open position.

In some embodiments, contaminants can be trapped and treated in or on a filter. The filter may be a high efficiency particle arresting (HEPA) filter or other particle capturing material that restricts the passage of particles or aerosols through the material. In some embodiments, it can be desirable to treat the particles on the filter so that the contaminants themselves do not degrade the performance of the filter. The particles can be treated so they are rendered inert and cannot cause harm if the particles come off the filter either in standard use or when replacing the filter. In some cases, materials on the filter can be treated by UV illumination and/or a catalyst that is part of the filter and arranged to oxidize the materials, e.g., at room temperature.

These and other aspects of the invention will be apparent from the following description and claims.

### BRIEF DESCRIPTION OF DRAWINGS

Aspects of the invention are described below with reference to the following drawings in which like numerals reference like elements, and wherein:

FIG. 1 shows a front view of a sanitizer apparatus in one embodiment.

FIG. 2 shows a front right perspective view of the FIG. 1 sanitizer apparatus.

FIG. 3 shows a front right perspective view of the FIG. 1 sanitizer apparatus with a front portion of the housing removed.

FIG. 4 shows a front view of the FIG. 1 sanitizer apparatus with the doors in an open position to expose a UV illumination source.

FIG. 5 is a cross sectional view along the line 5-5 in FIG. 4.

### DETAILED DESCRIPTION

It should be understood that inventive aspects are described herein with reference to certain illustrative embodiments and the figures. The illustrative embodiments described are not necessarily intended to show all inventive aspects, but rather are used to describe a few illustrative embodiments. Thus, inventive aspects are not intended to be construed narrowly in view of the illustrative embodiments. In addition, it should be understood that certain features may be used alone or in any suitable combination with other features. For example, features relating to positioning of a door within the apparatus housing in the open position can be combined, or not, with the feature of exposing a gap for air flow from the door opening to a filter when the doors are in an open position and/or with the feature of the door impeding air flow along an air flow path when in an open position.

Throughout this specification and in the claims, the term sanitizer apparatus is intended to relate to an apparatus for sanitizing, filtering, decontaminating, deodorizing, purifying, cleaning, conditioning, heating, humidifying, drying and/or otherwise treating an atmosphere and one or more surfaces within a space, such as a room.

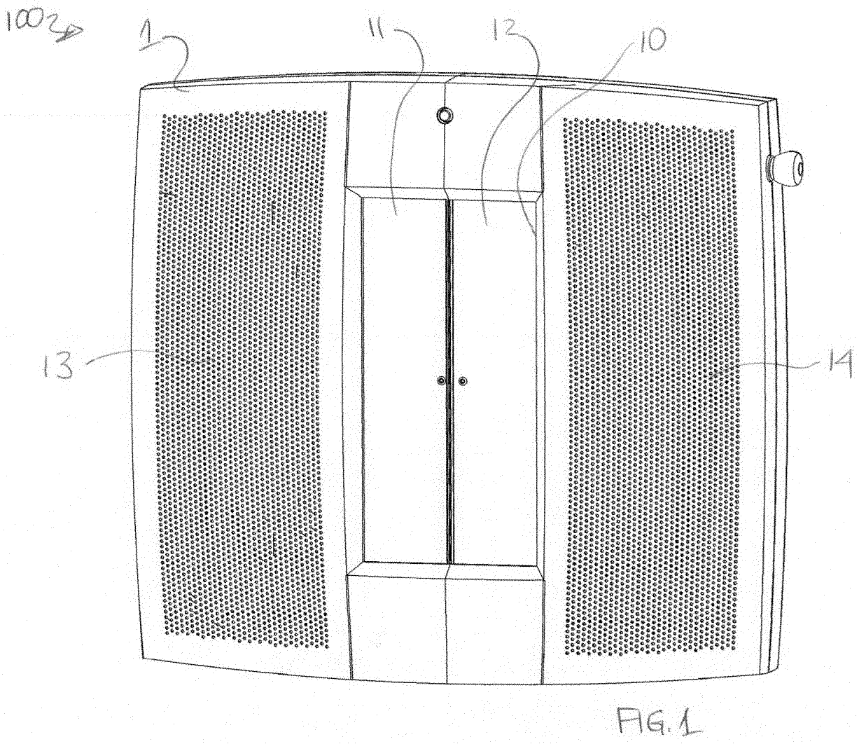

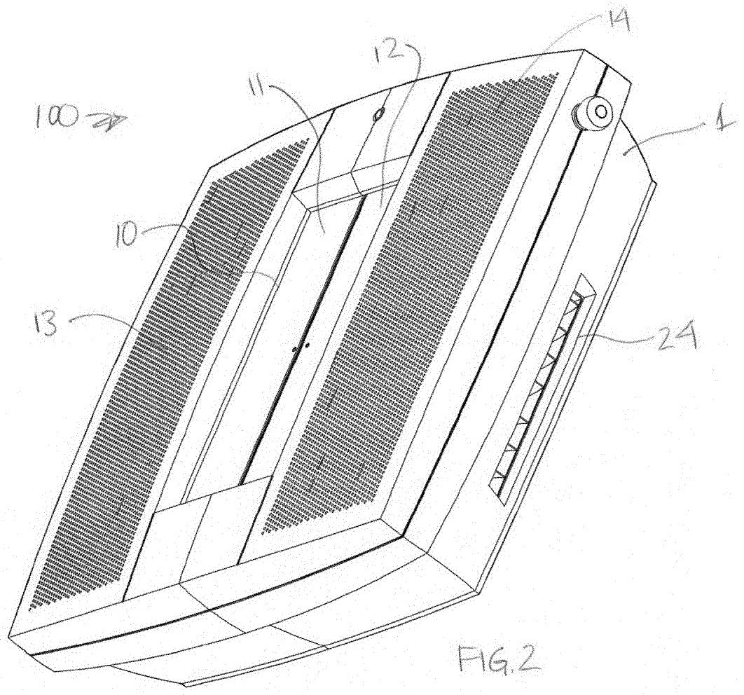

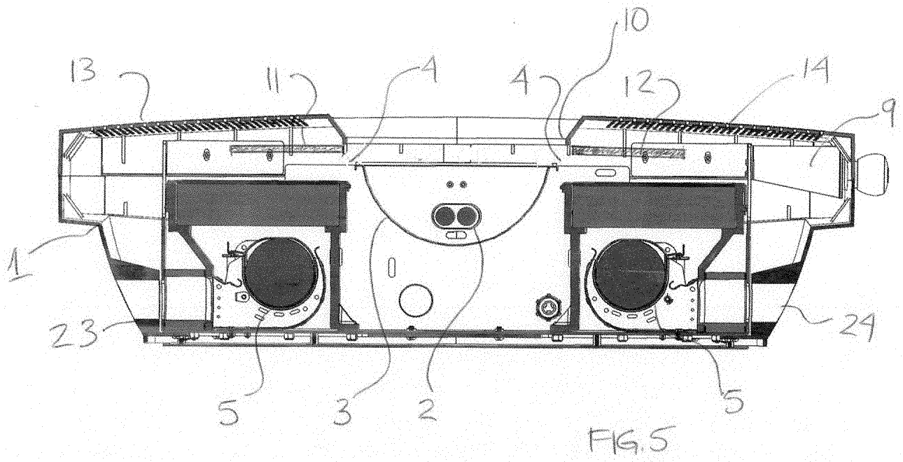

FIG. 1 is a front view and FIG. 2 is a front right perspective view of a sanitizer apparatus 100 including components that can be employed to achieve at least some objectives described herein. The apparatus 100 includes a housing 1 having first and second air inlets 13, 14 to receive air into the housing 1 and first and second air outlets 23, 24 to exhaust treated air. (The first air outlet 23 is visible in FIG. 5). In some embodiments, the apparatus 100 can have only one air inlet and/or one air outlet rather than two or more inlets/outlets as in other embodiments. First and second doors 11, 12 are mounted at a door opening 10 of the housing 1 and can be moved between a closed position shown in FIGS. 1 and 2 to an open position in which the doors 11, 12 are positioned away from the door opening 10. In some embodiments, the apparatus 100 can have only one door rather than two or more doors as in other embodiments.

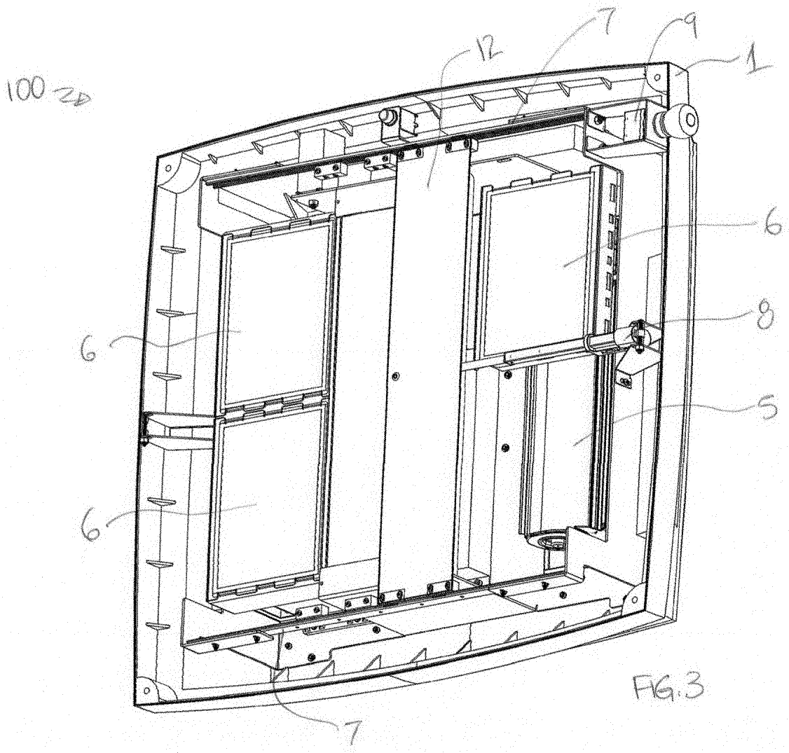

FIG. 3 shows the apparatus 100 with a front portion of the housing 1 removed to expose components that are positioned within the housing 1. One or more filters 6 are arranged in the housing 1 and configured to trap or otherwise remove particulate material from air moving along an air flow path. The filters 6 can be arranged in any suitable way, such as including activated charcoal, pleated paper or other porous sheet material (e.g., HEPA filters), metal or other screen material, catalyst material to oxidize contaminants (e.g., including TiO2, MnO2 or other materials to catalyze contaminants at room temperature or elevated temperatures), and so on. In short, one or more filters 6 can be provided and be configured to trap, capture, oxidize or otherwise remove contaminants, such as particulate matter, volatile or non-volatile compounds, etc. from air moving along the air flow path from an air inlet 13, 14 to an air outlet 23, 24. One or more air movers 5 are arranged to move air along an air flow path in the housing 1, e.g., from the air inlet 13, 14 to a filter 6 and then to the air outlet 23, 24. In some embodiments, the air movers 5 can be scroll or other fans, or other arrangements suitable to move air. One or more air movers 5 can be provided and can be positioned in any suitable way to move air along an air flow path, e.g., upstream or downstream of a filter 6, upstream or downstream of an air inlet 13, 14 or air outlet 23, 24, etc. A controller 9 can be configured to control operation of the components of the apparatus 100, such as the air mover 5, and can include any suitable hardware, software and other components to perform various component control and other functions described herein. For example, the controller 9 can include one or more door actuators 8 arranged to move the doors 11, 12 between open and closed positions. Such actuators 8 can be arranged in any suitable way, e.g., as solenoid or other electric motor actuated devices, hydraulic or pneumatic motors, etc., and can include any suitable linkages, coupling, etc. In some embodiments, the doors 11, 12 can move along a linear path between open and closed positions, e.g., along a pair of linear tracks 7 which guide the movement of the doors 11, 12. (Note the first door 11 is not shown in FIG. 3 for clarity.) In some embodiments, the doors 11, 12 can pivot or otherwise move along a rotational path between open and closed positions. When moving along a pivotal or other rotational path, the doors 11, 12 can move to an open position within the housing 1 or outside of the housing 1. As an example, the doors 11, 12 can be mounted by a hinge or other pivot at respective sides of the door opening 10 and can pivot from a closed position like that shown in FIG. 1 to an open position in which the doors 11, 12 are located outside of the housing 1 and at least partially over a respective air inlet 13, 14, e.g., so as to impede air flow into the air inlet 13, 14. Of course, the doors 11, 12 can move along a linear or other path to a position outside of the housing 1 and at least partially over an air inlet 13, 14. Pivotal movement of a door can be about a single pivot axis or about multiple pivot axes, e.g., as with a dual pivot or other multi-pivot hinge. In some embodiments, the doors 11, 12 can pivot inwardly, into the housing 1, from the closed to open position. In some cases, the doors 11, 12 can function as a reflector for the UV illumination source 2 when in the open position. As an example, the doors 11, 12 can pivot or otherwise move into a cavity where the UV illumination source 2 is located and be configured to reflect or otherwise direct light from the UV source to areas outside of the door opening 10. In some embodiments, movement of the doors 11, 12 can be complex, e.g., including both linear and pivotal movement. In some embodiments, regardless of how the doors 11, 12 move, with the doors 11, 12 in the closed position, air can be drawn into the air inlets 13, 14, through the filters 6 and exhausted out of the air outlets 23, 24 without being impeded by the doors 11, 12.

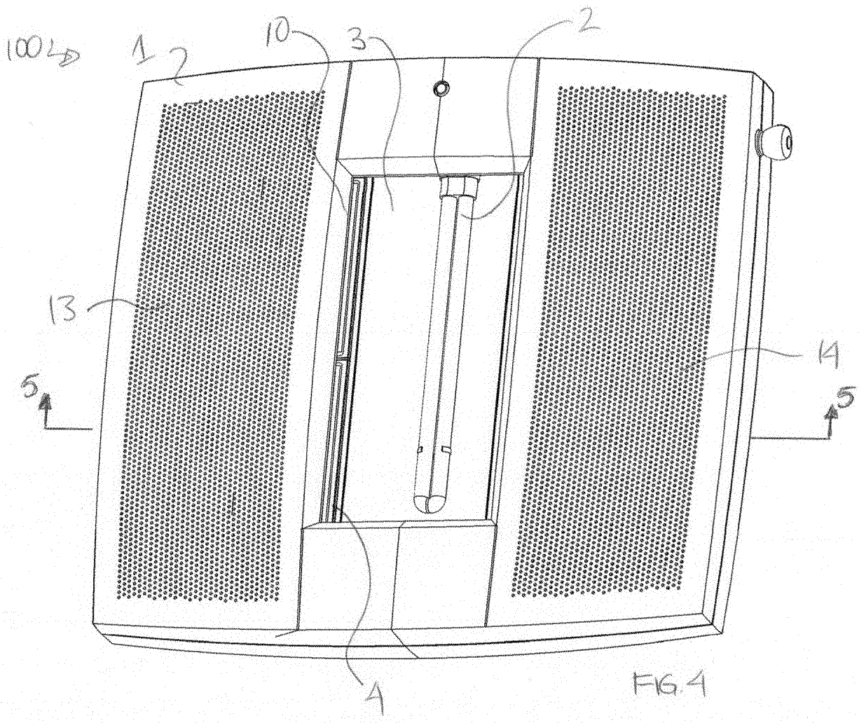

As can be seen in FIG. 4, when the doors 11, 12 are moved to an open position, a UV illumination source 2 in the housing can be exposed at the door opening 10. The UV illumination source 2 (e.g., including a UV bulb or LED array) can be configured to emit UV illumination suitable to sanitize air and/or surfaces exposed to the UV illumination. When the doors 11, 12 are in the closed position, the doors 11, 12 are configured to block UV illumination from the UV illumination source 2 from exiting the housing 1 through the door opening 10. However, when in the open position, UV illumination is permitted to exit the housing 1 through the door opening 10, e.g., to help sanitize air or surfaces around the apparatus 100. As an example, UV illumination can be used to sanitize floors, bed covers, desk or table surfaces, chairs, etc. in a room or other space where the sanitizing apparatus 100 is deployed. In some embodiments, the UV illumination source 2 is arranged to produce little or no ozone while emitting UV illumination suitable to inactivate microorganisms. A reflector 3 can be configured in the housing 1 to reflect UV illumination and/or direct UV illumination in desired ways to areas around the apparatus 100, e.g., to uniformly or otherwise illuminate a desired area of a particular shape and/or size. The reflector 3 can be movable and/or have movable components to scan UV illumination or otherwise direct UV illumination in a controllable way from the housing 1. In some embodiments, the reflector 3 can have a parabolic or otherwise curved shape and can be arranged so that the UV illumination source 2 is at a focal point or other suitable location with respect to optical properties of the reflector 3.

In some embodiments, the doors 11, 12 can be moved to an open position in which the doors 11, 12 are positioned within the housing 1 as shown in FIG. 4. (Note, however, that this is not required in all embodiments; in other embodiments the doors 11, 12 can be positioned outside of the housing 1 when in the open position.) In some cases where the doors 11, 12 are positioned within the housing 1 in the open position, the doors 11, 12 can be positioned between an air inlet 13, 14 and a filter 6 or other element along an air flow path. For example, the doors 11, 12 in the open position can be positioned adjacent an inner side of the housing, e.g., between the inner side of the housing and the filter 6. In some embodiments, the air inlets 13, 14 can be formed by a plurality of openings of the housing 1 that extend from an outer side of the housing 1 to the inner side of the housing 1. When in the open position, the doors 11, 12 can be positioned adjacent the plurality of openings at the inner side of the housing 1, e.g., between the plurality of openings and the filter 6.

In some embodiments, movement of a door between open and closed positions can cause air flow along an air flow path to be impeded. For example, with the doors 11, 12 in the closed position as shown in FIG. 1, the doors 11, 12 are positioned away from an air flow path so air can be free to move into the air inlet 13, 14 and flow unimpeded along the air flow path to a filter 6, through an air mover 5 and to the air outlet 23, 24. Also, when the doors 11, 12 are in the closed position, air and light are impeded or prevented from passing through the door opening 10. However, with the doors 11, 12 in the open position, e.g., as shown in FIGS. 4 and 5, the doors 11, 12 can be positioned to impede flow along an air flow path, e.g., positioned in the air flow path from the air inlet 13, 14 to a filter 6. In some embodiments, the doors 11, 12 can be positioned downstream of an air inlet 13, 14 to impede air flow as shown in FIG. 5, or can be positioned upstream of an air inlet 13, 14, e.g., outside of the housing and over an air inlet 13, 14 in the open position. In some embodiments, air flow along the air flow path from the air inlet 13, 14 can be completely blocked or impeded by a door 11, 12; in other embodiments air flow along the air flow path can be partially blocked or impeded, e.g., so air can flow around the door 11, 12 to a filter 6. Impeding the air flow along the air flow path from the air inlet 13, 14 can cause air to flow into the housing 1 along alternate flow paths than through an air inlet 13, 14. For example, in some embodiments when the doors 11, 12 are in the open position, air can flow through the door opening 10 and into the housing 1, e.g., to a filter 6. This can allow air to be treated by UV illumination as the air flows through the door opening 10, e.g., in a direction opposite to the direction in which UV illumination exits the door opening 10, and thereby help sanitize the air. That is, with the doors 11, 12 open, operation of the air mover 5 can cause air to be drawn through the door opening 10 and into a filter 6 or other part of the air flow path. While passing through the door opening 10, the air can be exposed to UV illumination (if the UV source is operating), thereby helping to sanitize the air. This can help keep the filter 6 clean or otherwise aid in sanitizing air.

In some embodiments, air flowing through the door opening 10 can flow into a gap 4 between a door 11, 12 and another portion of the apparatus 100, such as the reflector 3. That is, in some embodiments, the doors 11, 12 and the reflector 3 or other housing portion can be arranged so that a gap 4 is present between an inner side of the door 11, 12 and the reflector 3 or other housing portion so air flowing through the door opening 10 can flow through the gap 4 to the filter 6 or other portion of the air flow path. Thus, in some cases air that flows to a filter 6 can pass through both an air inlet 13, 14 and a gap 4 between a door 11, 12 and the reflector 3 or other housing portion at a cavity in which the UV illumination source 2 is located. In some cases, air flow through the gap 4 can help provide air to the filter 6 that is blocked or impeded by the doors 11, 12 when in the open position. As a result, an air flow rate to and through the filter 6 can be the same with the doors 11, 12 in the open and closed position for a given air mover 5 operation rate even though air may enter the housing 1 via different flow paths for the door open and closed positions. In some cases, an air flow rate through the filter 6 can be less when the doors 11, 12 are in the open position than in the closed position, e.g., air flow through the filters 6 can be zero when the doors 11, 12 are in the open position.

In some embodiments, while movement of a door to an open position can cause air flow along an air flow path to be impeded, the door need not itself impede or block the air flow. For example, a panel, louver, shutter or other element can be coupled to the door movement so that when the door moves to the open position, the panel or other element impedes air flow along an air flow path. In some cases, a door actuator 8 can be coupled to both a door and louver or other element so that when the door is moved to the open position, the louver or other element moves to impede air flow along an air flow path, e.g., by preventing air flow into an air inlet 13, 14. In some embodiments, a panel or other element can be coupled to the door so the panel or other element moves in response to door movement to the open position to an air flow impeding configuration. In some embodiments, movement of the door to the open position can cause the controller 9 to cause movement of a shutter or other element so that air flow is impeded, e.g., by controlling an actuator for the shutter to close the shutter when the door actuator is activated to open the door. Movement of a door to an open position and corresponding impeding of air flow along an air flow path can be coupled with control of the air mover, e.g., to adjust an air flow rate up or down from an air flow rate used while the door is closed.

In some embodiments, the housing 1 can be configured to be mounted on a ceiling of a room so that the front side of the housing 1 having the doors 11, 12, door opening 10 and air inlets 13, 14 faces downwardly toward the floor of the room. This arrangement can allow the apparatus 100 to draw air upwardly in the room toward the air inlets 13, 14 and exhaust cleaned air from the air outlets 23, 24 outwardly and away from the housing 1 in directions along the plane of the ceiling. This type of air flow can help circulate air in a room and aid in ensuring all air in the room is circulated through the apparatus 100. With the doors 11, 12 closed, air flow through the air inlets 13, 14 and along a respective air flow path can be unimpeded by the doors 11, 12. Mounting the apparatus 100 on a ceiling can also allow the apparatus 100 to emit UV illumination from the UV illumination source 2 (with the doors 11, 12 in the open position so the door opening 10 is unobstructed) so the UV illumination is incident on surfaces of the room below the ceiling, such as floors, bedclothes, desk or table tops, chairs, furnishings, etc. This can help sanitize surfaces, e.g., neutralizing microbes such as viruses and bacteria and/or oxidizing or otherwise sanitizing materials on the surfaces such as grease, oils, dust, and so on. The UV illumination source 2 may operate simultaneously with the air mover 5 whether the doors 11, 12 are open or closed, or can operate at separate times, e.g., only the air mover 5 is operated when the doors 11, 12 are closed and only the UV illumination source 2 is operated when the doors 11, 12 are open. Operation of the UV illumination source 2 and the air mover 5 while the doors 11, 12 are closed can help expose air entering the housing 1 via the air inlets 13, 14 to UV illumination since the UV illumination is blocked from exiting the housing 1 via the door opening 10. In some cases, the UV illumination source 2 can illuminate portions of the filter 6, e.g., to aid in cleaning the filter 6, when the doors 11, 12 are closed. As an example, when the doors 11, 12 are closed, one or more portions of the reflector 3 can move to permit UV illumination to reach the filter 6, and when the doors 11, 12 are open, the reflector 3 can be moved or otherwise arranged to direct UV light through the door opening 10.

In some embodiments where the apparatus 100 is employed in a room or other space where people or animals are present, the controller 9 can operate to help ensure that the UV illumination source 2 does not operate with the doors 11, 12 in the open position while people or animals are present. For example, the controller 9 can include a motion sensor (e.g., a camera and associated image analysis software, passive infrared detector, ultrasonic motion detector, etc.) arranged to detect the presence of people or animals and can prevent operation or shut down the UV illumination source 2 if people or animals are detected. In some embodiments, the controller 9 can include a door, window or other entry point sensor that can detect whether a door, window or other entry point to a room or other space in which the apparatus 100 is deployed is open or closed. The controller 9 can prevent operation of the UV illumination source 2 with the doors 11, 12 open when a door, window, etc. is open to the room or other space in which the apparatus 100 is employed. In some embodiments, the controller 9 can include a clock or other timer so the UV illumination source 2 is operated during times of the day or week when people or animals are less likely to be present. In short, the controller 9 can be configured (e.g., by suitable programming or other instructions) to help minimize the chance that a person or animal will be exposed to UV illumination. Note that in some embodiments the controller 9 can be in communication (e.g., by wired and/or wireless communication links) to a building or room controller that includes or otherwise receives sensor information such as motion in the building or room, door/window opening or closing, etc. Such a building or room controller can operate to control any suitable functions, such as security, HVAC, lighting, power, etc., for a building and can provide instructions or other information to the apparatus 100 to operate or that can otherwise be used by the controller 9 to determine whether conditions are safe for operation of the air mover 5 and/or UV illumination source 2. Thus, the sanitizing apparatus 100 can operate under the control of a building or room controller and/or can operate more autonomously based on information received from the building or room controller (or not).

Those skilled in the art will appreciate that the conception, upon which this disclosure is based, may readily be utilized as a basis for the designing of other structures, methods, and systems for carrying out the several purposes of the disclosed subject matter. It is important, therefore, that the claims be regarded as including such equivalent constructions insofar as they do not depart from the spirit and scope of the disclosed subject matter. For example, the term “air” is used in general in this document and it can be interpreted to include both natural air and/or any gaseous or vaporous matter.

Operation of the apparatus components may be controlled by the controller 9, which may include a programmed processor and/or other data processing device along with suitable software or other operating instructions, one or more memories (including non-transient storage media that may store software and/or other operating instructions), temperature and other sensors, pressure sensors, input/output interfaces (such as a user interface on the housing 1, indicator lights or other displays, etc.), communication buses or other links, a display, switches, relays, triacs, or other components necessary to perform desired input/output or other functions. A user interface may be arranged in any suitable way and include any suitable components to provide information to a user and/or receive information from a user, such as buttons, a touch screen, a voice command module (including a microphone to receive audio information from a user and suitable software to interpret the audio information as a voice command), a visual display, one or more indicator lights, a speaker, and so on.

Having thus described several aspects of at least one embodiment of this invention, it is to be appreciated various alterations, modifications, and improvements will readily occur to those skilled in the art. Such alterations, modifications, and improvements are intended to be part of this disclosure and are intended to be within the spirit and scope of the invention. Accordingly, the foregoing description and drawings are by way of example only.

1. An air and surface treatment apparatus, comprising:
a housing having an air inlet, an air outlet and an air flow path through the housing from the air inlet to the air outlet;
an air mover in the housing and configured to move air from the air inlet to the air outlet along the air flow path;
a filter arranged in the air flow path and configured to remove particulate material from air moving along the air flow path;
a UV illumination source in the housing and configured to emit UV illumination suitable to sanitize air and/or surfaces exposed to the UV illumination; and
a door mounted at a door opening of the housing and configured to move between a closed position in which the door is configured to block UV illumination emitted by the UV illumination source from exiting the housing and an open position in which the UV illumination is permitted to exit the housing through the door opening, wherein movement of the door from the closed position to the open position configures the apparatus to impede flow of air along the air flow path.

2. The apparatus of claim 1, wherein in the closed position the door is positioned away from the air flow path and in the open position the door is positioned to impede flow of air along the air flow path.

3. The apparatus of claim 1, wherein the door is configured to move along a linear or rotational path between the open and closed positions.

4. The apparatus of claim 1, wherein in the open position the door is positioned between the air inlet and the filter.

5. The apparatus of claim 4, wherein the housing includes a plurality of openings that form the air inlet, the plurality of openings extending from an outer side of the housing to an inner side of the housing, and wherein in the open position, the door is positioned between the plurality of openings and the air filter.

6. The apparatus of claim 1, wherein the housing includes a plurality of openings that form the air inlet, the plurality of openings extending from an outer side of the housing to an inner side of the housing, and wherein in the open position, the door is positioned adjacent the inner side of the housing and the plurality of openings.

7. The apparatus of claim 1, wherein in the open position, the door is positioned within the housing.

8. The apparatus of claim 1, wherein in the open position, the door is configured to permit flow of air through the door opening and to the filter, and in the closed position, the door is configured to impede flow of air through the door opening.

9. The apparatus of claim 1, wherein the door is a first door, the apparatus further comprising a second door mounted at a door opening of the housing and configured to move between a closed position in which the second door is configured to block UV illumination emitted by the UV illumination source from exiting the housing and an open position in which the UV illumination is permitted to exit the housing through the door opening.

10. The apparatus of claim 9, wherein the first and second doors are arranged to close the door opening when the first and second doors are in the closed position and to open the door opening when the first and second doors are in the open position.

11. The apparatus of claim 10, wherein the first and second doors move away from each other along a linear path when moving from the closed position to the open position.

12. The apparatus of claim 1, wherein the filter is a first filter, the air inlet is a first air inlet and the air flow path is a first air flow path, the apparatus further comprising a second filter, a second air inlet and a second air flow path, wherein in the closed position the second door is positioned away from the second air flow path and in the open position the second door is positioned to impede flow of air along the second air flow path from the second air inlet to the second filter.

13. The apparatus of claim 1, wherein in the open position, the first and second doors are configured to permit flow of air through the door opening and to the first and second filters, and in the closed position, the first and second doors are configured to impede flow of air through the door opening.

14. An air and surface treatment apparatus, comprising:
a housing having an air inlet, an air outlet and an air flow path through the housing from the air inlet to the air outlet;
an air mover in the housing and configured to move air from the air inlet to the air outlet along the air flow path;
a filter arranged in the air flow path and configured to remove particulate material from air moving along the air flow path;
a UV illumination source in the housing and configured to emit UV illumination suitable to sanitize air and/or surfaces exposed to the UV illumination; and
a door mounted at a door opening of the housing and configured to move between a closed position in which the door is configured to block UV illumination emitted by the UV illumination source from exiting the housing and an open position in which the UV illumination is permitted to exit the housing through the door opening, wherein in the closed position the door is configured to impede air flow into the door opening and in the open position the door is positioned to permit flow of air through the door opening and into a gap on an inner side of the door.

15. The apparatus of claim 14, wherein air flow through the door opening and into the gap is driven by the air mover and air flow into the gap passes to the filter.

16. The apparatus of claim 14, wherein the gap is arranged between the inner side of the door and a reflector for the UV illumination emitted by the UV illumination source.

17. The apparatus of claim 14, wherein the housing includes a plurality of openings that form the air inlet, the plurality of openings extending from an outer side of the housing to an inner side of the housing, and wherein in the open position the door is configured to impede flow of air along the air flow path.

18. An air and surface treatment apparatus, comprising:
a housing having an air inlet, an air outlet and an air flow path through the housing from the air inlet to the air outlet;
an air mover in the housing and configured to move air from the air inlet to the air outlet along the air flow path;
a filter arranged in the air flow path and configured to remove particulate material from air moving along the air flow path;
a UV illumination source in the housing and configured to emit UV illumination suitable to sanitize air and/or surfaces exposed to the UV illumination; and
a door mounted at a door opening of the housing and configured to move between a closed position in which the door is configured to block UV illumination emitted by the UV illumination source from exiting the housing and an open position in which the UV illumination is permitted to exit the housing through the door opening, wherein in the open position the door is positioned within the housing.

19. The apparatus of claim 18, wherein the housing includes a plurality of openings that form the air inlet, the plurality of openings extending from an outer side of the housing to an inner side of the housing, and wherein in the open position the door is configured to impede flow of air along the air flow path.

20. The apparatus of claim 18, wherein the door is a first door, the filter is a first filter, the air inlet is a first air inlet and the air flow path is a first air flow path, the apparatus further comprising a second door mounted at the door opening and movable between open and closed positions, a second filter, a second air inlet and a second air flow path, wherein in the open position the second door is positioned within the housing.

21. The apparatus of claim 20, wherein in the open position the first and second doors are positioned to impede flow of air along the first and second air flow paths, respectively, from the first and second air inlet to the first and second filter.

22. The apparatus of claim 19, wherein the door is configured to move along a linear or rotational path between the open and closed positions
